(19) United States
(12) Patent Application Publication
(10) Pub. No.: US 2008/0003259 A1
(43) Pub. Date: Jan. 3, 2008

(54) Modification of surfaces of polymeric articles by Michael addition reaction

(76) Inventors: Joseph C. Salamone, Boca Raton, FL (US); Yu-Chin Lai, Pittsford, NY (US); Weihong Lang, Penfield, NY (US); Wenyan Yan, Fairport, NY (US)

Correspondence Address: Bausch & Lomb Incorporated, One Bausch & Lomb Place, Rochester, NY, 14604-2701, US

(21) Appl. No.: 11/480,243

(22) Filed: Jun. 30, 2006

(51) Int. Cl. A61F 2/02

(52) U.S. Cl. 424/427; 424/428

(57) ABSTRACT

A medical device having an increased surface hydrophilicity comprises a coating polymer comprising units of a polymerizable hydrophilic compound that is attached to the surface of the medical device via the Michael addition reaction. The coating polymer can be applied to a medical device comprising a hydrogel material. The attachment of the coating polymer may be enhanced by increasing the population of the medical-device surface functional groups before contacting the medical device with the coating polymer.

### BACKGROUND OF THE INVENTION

The present invention relates to modification of surfaces of polymeric articles by the Michael addition reaction. In particular, the present invention relates to medical devices having surfaces modified by the Michael addition reaction.

Advances in the chemistry of materials for medical devices have increased the comfort for their extended use in a body environment. Furthermore, extended use of medical devices, such as ophthalmic lenses, has become increasingly favored due to the availability of soft contact lenses having high oxygen permeability (e.g., exhibiting high Dk values greater than 80) and/or high water content. Such lenses are increasingly made of silicone-containing materials. Although these materials have some desirable properties for ophthalmic applications, they tend to have relatively hydrophobic surfaces that have a high affinity for lipids and proteins. Accumulation of these materials can interfere with the clarity of the lens and the comfort of the wearer. On the other hand, hydrophilic surfaces tend to limit the adsorption onto and absorption into ophthalmic lenses of tear lipids and proteins and allow the lenses to move relatively freely on the eye, thus providing increased comfort to the wearer.

A known method for modifying the surface hydrophilicity of a relatively hydrophobic ophthalmic device, such as a contact lens, is through the use of a plasma treatment. Plasma treatment techniques are disclosed, for example, in PCT Publications WO 96/31792 to Nicolson et al., WO 99/57581 to Chabrececk et al., and WO 94/06485 to Chatelier et al. In the Chabrececk et al. application, photoinitiator molecules are covalently bound to the surface of the article after the article has been subjected to a plasma treatment which provides the surface with functional groups. A layer of polymerizable macromonomer is then coated onto the modified surface and heat or radiation is applied to graft polymerize the macromonomer to form the hydrophilic surface. However, it may be difficult to provide an effective number of photoinitiators on the surface to effect a strong attachment of the resulting polymer.

Other methods of permanently altering the surface properties of polymeric biomaterials, such as contact lenses, have been developed. Some of these techniques include Langmuir-Blodgett deposition, controlled spin casting, chemisorptions, and vapor deposition. Examples of Langmuir-Blodgett layer systems are disclosed in U.S. Pat. Nos. 4,941,997; 4,973,429; and 5,068,318. Like plasma treatments, these techniques are not cost-effective methods that may easily be incorporated into automated production processes for making biomedical devices such as contact lenses.

Another method of producing a hydrophilic surface is discussed in U.S. Pat. No. 6,926,965. The method is carried out in a layer-by-layer (LbL) fashion, which involves consecutively dipping a substrate into oppositely charged polymeric materials until a coating of a desired thickness is formed.

These prior-art methods are tedious. As a result, the manufacturing costs for the finished devices can be high.

Therefore, there is a continued need to provide medical devices, such as ophthalmic lenses, that have improved hydrophilic surfaces and are compatible with physiological environment, and improved methods for making them.

### SUMMARY OF THE INVENTION

In general, the present invention provides a method for modifying surfaces of polymeric articles by the Michael addition reaction (sometimes also referred to in the art as “Michael reaction”). The present invention also provides such polymeric articles having surfaces comprising attached polymeric materials.

In one aspect, the polymeric articles are medical devices that can provide higher level of performance quality and/or comfort to the users.

In one aspect, the present invention provides a medical device having a polymer coating on a surface of the medical device.

In another aspect, the polymer coating comprises a hydrophilic polymer coating.

In still another aspect, the hydrophilic polymer coating is attached directly or indirectly to the surface of the medical device.

In still another aspect, the coating comprises a coating polymer covalently attached directly or indirectly to the surface of the medical device.

In yet another aspect, the coating polymer is attached to the surface of the medical device by the Michael addition reaction.

In still a further aspect, the medical device and the coating polymer have complementary functional groups that participate in the Michael addition reaction.

In still another aspect, the medical devices are ophthalmic devices.

In yet another aspect, the medical devices are contact lenses.

In still another aspect, the medical devices have reduced contact angles compared to those that do not have a polymeric coating of the present invention.

In a further aspect, the present invention provides a method of making a medical device that has a hydrophilic surface. The method comprises: (a) providing the medical device having at least a medical-device surface functional group; (b) providing a polymer having at least a hydrophilic moiety and at least a polymer functional group capable of interacting with said at least a medical-device surface functional group in the Michael addition reaction; and (c) contacting the medical device with the polymer at a condition sufficient to produce the medical device having an increased surface hydrophilicity.

Other features and advantages of the present invention will become apparent from the following detailed description and claims.

### DETAILED DESCRIPTION OF THE INVENTION

In general, the present invention provides a method for modifying surfaces of polymeric articles by the Michael addition reaction. The present invention also provides such polymeric articles having surfaces comprising attached polymeric materials.

In one aspect, the polymeric materials (also herein sometimes referred to as coating polymeric materials) are attached to the surfaces of the polymeric articles by the Michael addition reaction, which is the nucleophilic addition to the α-β double bond conjugate to an electron-withdrawing group.

In one aspect, a coating polymeric material of the present invention comprises one or more α-β double bonds conjugate to electron-withdrawing groups (such as carbonyl or sulfonyl group), and the surfaces of the polymeric article comprise a plurality of nucleophilic groups.

In another aspect, the surfaces of the polymeric article comprise a plurality of α-β double bonds conjugate to electron-withdrawing groups (such as carbonyl or sulfonyl group), and a coating polymeric material of the present invention comprises one or more nucleophilic groups.

Non-limiting examples of nucleophilic groups are amines, thiols, hydroxyl, hydroxylamines, hydrazines, guanadines, imines, phosphines, and carbanions.

In one embodiment, the nucleophilic groups comprise the primary amine group (—NH₂), the secondary amine groups (—NHR), the tertiary amine groups (—NR¹R²), wherein R, R¹, and R² are monovalent groups, such as, for example, monovalent C₁₋₁₀ aliphatic hydrocarbon groups or alkylaryl groups, or aromatic tertiary amino groups (such as pyridine); and the coating polymeric material comprises the acrylate group. When the nucleophilic group comprises a tertiary amino group, the attachment reaction is advantageously carried out in the presence of a hydrogen donor.

In another aspect, the polymeric material of the article comprises the nucleophilic groups, some of which are exposed on the surface of the article.

In still another aspect, the nucleophilic groups are created on the surface of the polymeric article, such as by implantation of reactive moieties at the surface of such an article, which reactive moieties comprise such nucleophilic groups or by reaction of the surface material with a reagent to result in such nucleophilic groups.

In still another aspect, α-β double bonds conjugate to an electron-withdrawing groups are formed on the surfaces of the polymeric article by reacting the same with reactive compounds having such double bonds.

In yet another aspect, the polymeric article is a medical device. In one embodiment, the medical device is an ophthalmic device. In another embodiment, the ophthalmic device is a contact lens.

In a further aspect, the medical device comprises a siloxanyl-based polymer. The term “siloxanyl-based” means comprising a silicon-oxygen-silicon bond. Suitable siloxanyl-based polymers are disclosed below.

In still a further aspect, the present invention provides medical devices comprising hydrophilic surfaces and/or reduced dehydration rates and methods for making these devices.

A method of making a medical device that has a hydrophilic surface comprises: (a) providing the medical device having at least a medical-device surface functional group; (b) providing a polymer having at least a hydrophilic moiety and at least a polymer functional group that is capable of interacting with said at least a medical-device surface functional group in the Michael addition reaction; and (c) contacting the medical device with the polymer at a condition sufficient to produce the medical device having an increased surface hydrophilicity.

In one embodiment, the step of providing the medical device having at least a medical-device surface functional group comprises creating the surface functional group by implantation of moieties that comprise the surface functional group. The implantation is effected at or in the surface of the medical device. In another embodiment, the step of providing the medical device having at least a medical-device surface functional group comprises creating the surface functional group by reacting the material of the surface of the medical device with a suitable reagent to form the surface functional group. In still another embodiment, the suitable reagent is an oxidizing agent. In yet another embodiment, the step of reacting comprises exposing the surface to plasma containing an oxidizing agent, such as an oxygen-containing species, ammonia, or amine.

In one embodiment of the present invention, wherein a contact lens comprises surface amino group, and the coating polymer comprises acrylate-terminated polyvinylpyrrolidone, the surface-modified contact lens is produced according to Scheme 1, wherein n is a positive integer. In one embodiment, n can range from about 5 to about 1000, or from about 10 to 800, or from about 10 to about 600, or from about 20 to about 500.

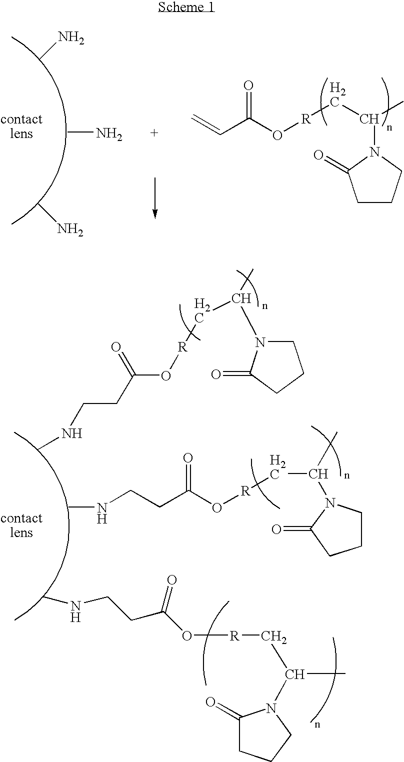

wherein R is a direct bond or a divalent group selected from the group consisting of C₁₋₁₀ saturated and unsaturated hydrocarbon groups, C₁₋₁₀ saturated and unsaturated hydrocarbon groups having one or more heteroatoms therein, C₃₋₁₀ cyclic hydrocarbon groups, C₃₋₁₀ heterocyclic groups, C₆₋₃₆ aryl groups, and C₆₋₃₆ heteroaryl groups.

In one aspect, the medical devices have increased surface lubricity.

In another aspect, the medical devices of the present invention provide higher level of performance quality and/or comfort to the users due to their hydrophilic or lubricious surfaces. In one embodiment, the medical devices are contact lenses, such as extended-wear contact lenses. Hydrophilic surfaces of such contact lenses substantially prevent or limit the adsorption of tear lipids and proteins on, and their eventual absorption into, the contact lenses, thus preserving the clarity of the contact lenses, and in turn preserving their performance quality and providing a higher level of comfort to the wearer.

In still another aspect, the present invention provides a medical device having a hydrophilic polymer coating that is attached to a surface of the medical device by the Michael addition reaction at surface functional groups. In one embodiment, the hydrophilic polymer coating comprises a plurality of hydrophilic moieties, which may be the same or different, and at least a polymer functional group that is capable of reacting with the surface functional groups in the Michael addition reaction. In one embodiment, such a polymer functional group comprises an α-β double bond conjugate to an electron-withdrawing group. In one embodiment, such an electron-withdrawing group is a carbonyl group. In another embodiment, such an electron-withdrawing group is a sulfonyl group.

In another aspect, the coating polymer comprises monomeric units selected from the group consisting of N,N-dimethylacrylamide, polymerizable polyhydric alcohols, polymerizable alkylene oxides, polymerizable carboxylic acids, derivatives thereof, combinations thereof, and mixtures thereof.

In another embodiment of the present invention, wherein a contact lens comprises surface amino group, and the coating polymer comprises methacrylate-terminated poly(ethylene glycol), the surface-modified contact lens is produced according to Scheme 2, wherein n is a positive integer. In one embodiment, n can range from about 5 to about 1000, or from about 10 to 800, or from about 10 to about 600, or from about 20 to about 500.

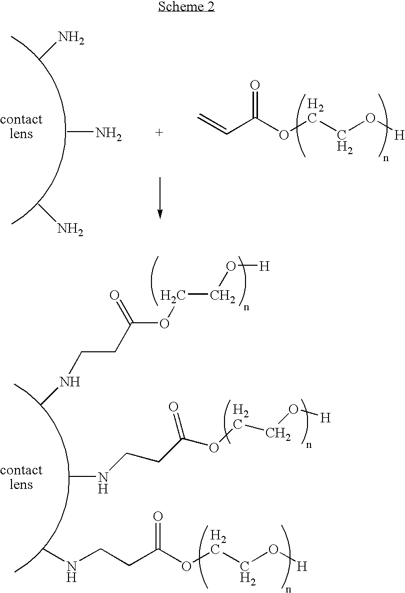

In yet another aspect, the polymerizable polyhydric alcohols comprise a material other than polymerizable poly(alkylene glycol) (or poly(oxyalkylene)) and derivatives thereof. In one embodiment, the polymerizable polyhydric alcohols are other than polymerizable poly(ethylene glycol) or polymerizable poly(propylene glycol). Non-limiting examples of such polymerizable polyhydric alcohols include glycerol (meth)acrylate, erythritol (meth)acrylate, xylitol (meth)acrylate, sorbitol (meth)acrylate, derivatives thereof, combinations thereof, or mixtures thereof. The term “(meth)acrylate” means methacrylate or acrylate. In one embodiment, the (meth)acrylate is mono(meth)acrylate. In another embodiment, di(meth)acrylate or a mixture of mono(meth)acrylate and di(meth)acrylate may be used.

In one embodiment, the coating polymer comprises polysaccharides (such as hyaluronic acid or hydroxypropylmethyl cellulose) that have at least a terminal functional group that is capable of participating in the Michael addition reaction.

In another embodiment, the coating polymer comprises a macromonomer having a formula of

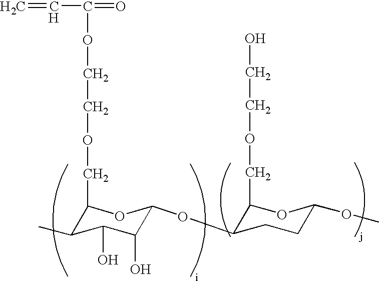

wherein i and j are independent integers. For example, 2≦i, j≦1000, or 10≦i, j ≦800, or 10≦i, j≦600, or 20≦i, j≦500.

In still another embodiment, the coating polymer can comprise carboxylic acids that are selected from alkenoic acids comprising 4 to and including 10 carbon atoms. In yet another embodiment, the alkenoic acids are selected from the group consisting of maleic acid, fumaric acid, itaconic acid, derivatives thereof (such as maleic anhydride, fumaric anhydride, or itaconic anhydride), combinations thereof, and mixtures thereof. The term “carboxylic acids” also includes compounds that are capable of being converted into carboxylic acids, such as, for example, vinyidimethyloxozalone (“VDMO”).

In still another embodiment, the coating polymer is attached to the surface of the medical device through an intermediate compound or linking compound or an intermediate polymer (or also herein sometimes called “linking polymer”) that has functional groups capable of interacting with functional groups on the surface of the medical device and functional groups of the coating polymer. Thus, the intermediate compound acts to couple the coating polymer to the surface of the medical device through the Michael addition reaction. For example, the intermediate compound or polymer can comprise terminal amino groups, which are capable of forming bonds with functional groups on the surface of the medical device and with the conjugate unsaturated bond at the end of the hydrophilic coating polymer. When the intermediate compound or polymer has a terminal amino group, it may be desirable to have no other functional groups in such intermediate compound or polymer that can react with the terminal amino group.

In another embodiment of the present invention, wherein a contact lens comprises surface carboxylic group, the coating polymer comprises acrylate-terminated poly(vinylpyrrolidone), and the linking compound is ethylene diamine, the surface-modified contact lens is produced according to Scheme 3, wherein n is a positive integer. In one embodiment, n can range from about 5 to about 1000, or from about 10 to 800, or from about 10 to about 600, or from about 20 to about 500, and R is a direct bond or a divalent group selected from the group consisting of C₁₋₁₀ saturated and unsaturated hydrocarbon groups, C₁₋₁₀ saturated and unsaturated hydrocarbon groups having one or more heteroatoms therein, C₃₋₁₀ cyclic hydrocarbon groups, C₃₋₁₀ heterocyclic groups, C₆₋₃₆ aryl groups, and C₆₋₃₆ heteroaryl groups.

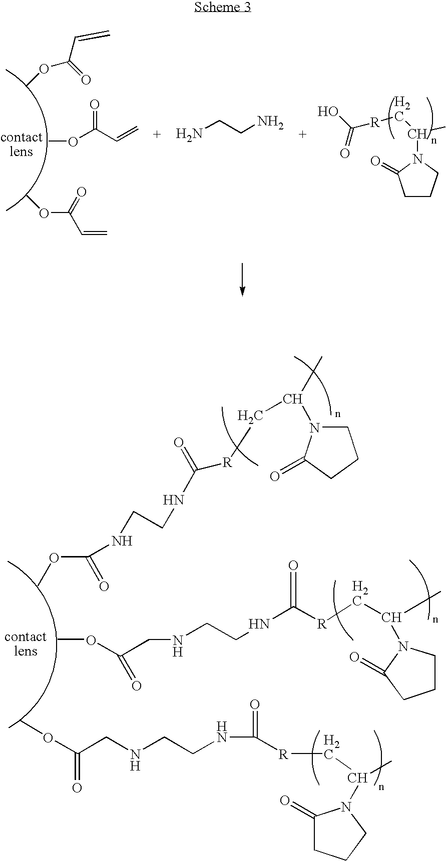

In a further embodiment, the medical device has a polymer coating consisting or consisting essentially of units of vinylpyrrolidone.

In one aspect, the surface treatment of the medical device can be carried out, for example, at about room temperature or under autoclave condition. The medical device is immersed in a solution comprising the coating polymer. Alternatively, the medical device is immersed in a solution comprising the coating polymer and the linking compound (or linking polymer). Thus, in one aspect, the medical device comes into contact with the linking compound and the coating polymer substantially simultaneously. In another aspect, the medical device is immersed in a solution comprising the linking compound. Then, after some elapsed time, the coating polymer is added to the solution in which the medical device is still immersed. In one embodiment of the method of treatment, the solution is aqueous. In another embodiment, the solution comprises a polar organic solvent, such as methanol or ethanol.

In another aspect, the medical device comprises a polymeric material and the nucleophilic functional groups on the surface thereof are parts of units of the polymeric material. For example, hydrogel polymers of contact lens typically comprise hydrophilic monomeric units, such as 2-hydroxyethyl methacrylate, which provides nucleophilic hydroxyl surface groups. Alternatively, a hydrogel polymer can comprise a suitable amount of a precursor of 2-aminoethyl methacrylate, which provides nucleophilic amino surface groups after the manufactured lens is neutralized.

In another aspect, the surface of the medical device can be treated with a plasma discharge or corona discharge to increase the population of reactive surface groups. The type of gas introduced into the treatment chamber is selected to provide the desired type of reactive surface groups. For example, hydroxyl surface groups can be produced with a treatment chamber atmosphere comprising water vapor or alcohols. Carboxyl surface groups can be generated with a treatment chamber comprising oxygen or air or another oxygen-containing gas. Ammonia or amines in a treatment chamber atmosphere can generate amino surface groups. Sulfur-containing gases, such as organic mercaptans or hydrogen sulfide, can generate the mercaptan group on the surface. A combination of any of the foregoing gases also can be used in the treatment chamber. Methods and apparatuses for surface treatment by plasma discharge are disclosed in, for example, U.S. Pat. Nos. 6,550,915 and 6,794,456, which are incorporated herein in their entirety by reference. Such a step of treatment with a discharge can be carried out before the treated device is contacted with a medium containing the coating polymer.

In another aspect, the surfaces of the polymeric article comprise a plurality of α-β double bonds conjugate to electron-withdrawing groups (such as carbonyl or sulfonyl group), and a coating polymeric material of the present invention comprises one or more nucleophilic groups. In one embodiment, the plurality of α-β double bonds conjugate to electron-withdrawing groups can be generated on the surfaces of the polymeric article by reacting the article with a compound that has a moiety that comprises such an α-β double bond. For example, a polymeric article the surfaces of which have a plurality of hydroxyl groups can be reacted with acryloyl chloride, methacryloyl chloride, fumaroyl chloride, or itaconyl chloride to generate a plurality of α-β double bonds conjugate to carbonyl electron-withdrawing groups. The polymeric article thus treated can then be exposed to a hydrophilic coating polymeric material comprising at least a nucleophilic group to form a hydrophilic coating thereon, as shown in Scheme 4, wherein the hydrophilic coating comprises a plurality of polyethylene glycol units.

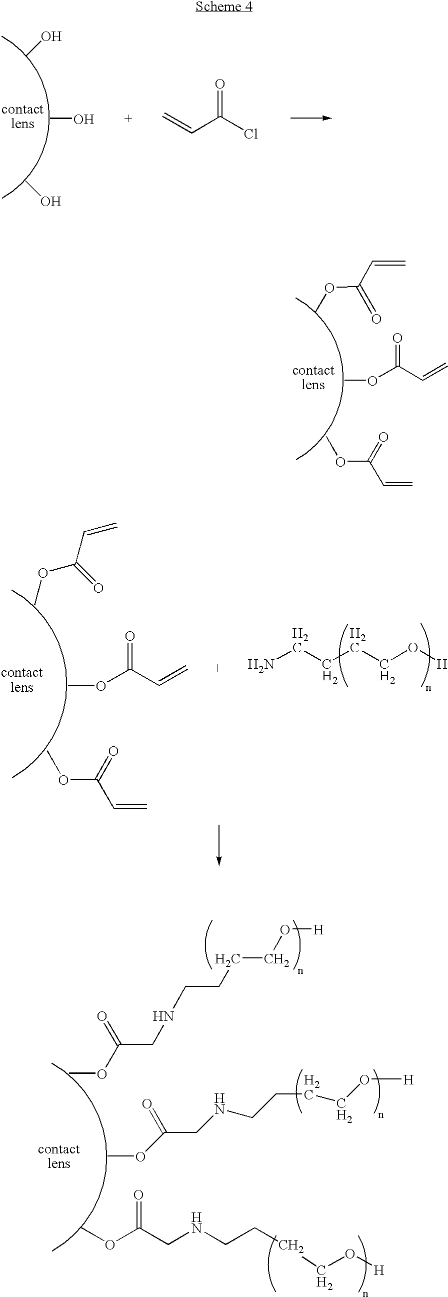

In another embodiment, an acryloyl-functionalized contact lens, as disclosed above, can be treated with hydrophilic poly(vinylpyrrolidone) to produce a hydrophilic coating as shown in Scheme 5.

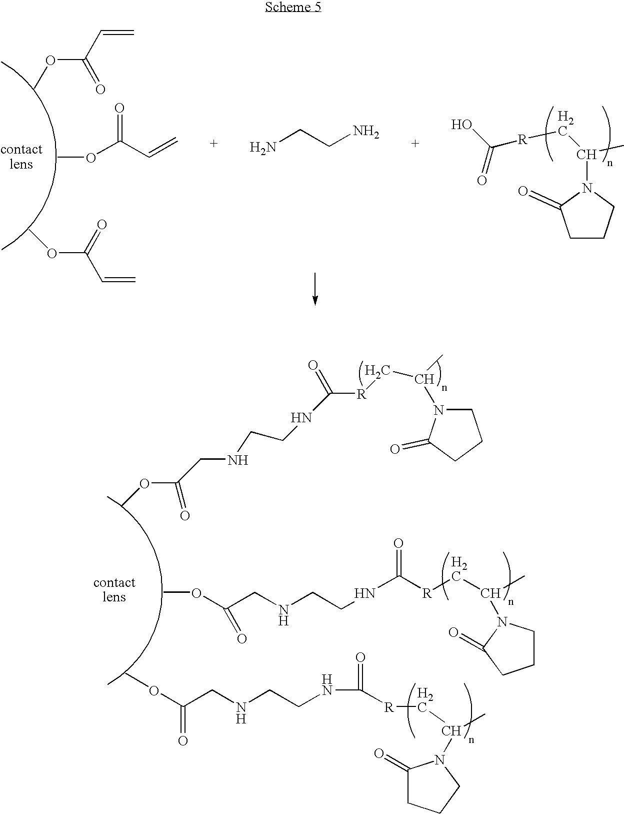

Medical devices comprising a wide variety of polymeric materials, including hydrogel and non-hydrogel materials, can be made to have hydrophilic surfaces. In general, non-hydrogel materials are hydrophobic polymeric materials that do not contain water in their equilibrium state. Typical non-hydrogel materials comprise silicone acrylics, such as those formed from bulky silicone monomer (e.g., tris(trimethylsiloxy)silylpropyl methacrylate, commonly known as “TRIS” monomer), methacrylate end-capped poly(dimethylsiloxane) prepolymer, or silicones having fluoroalkyl side groups. On the other hand, hydrogel materials comprise hydrated, cross-linked polymeric systems containing water in an equilibrium state. Hydrogel materials contain about 5 weight percent water or more (up to, for example, about 80 weight percent). Non-limiting examples of materials suitable for the manufacture of medical devices, such as contact lenses, are herein disclosed.

Hydrogel materials for medical devices, such as contact lenses, can comprise a hydrophilic monomer, such as, HEMA, methacrylic acid (“MMA”), acrylic acid (“AA”), methacrylamide, acrylamide, N,N′-dimethylmethacrylamide, or N,N′-dimethylacrylamide; copolymers thereof; hydrophilic prepolymers, such as poly(alkylene oxide) having varying chain length, functionalized with polymerizable groups; and/or silicone hydrogels comprising siloxane-containing monomeric units and at least one of the aforementioned hydrophilic monomers and/or prepolymers. Hydrogel materials also can comprise a cyclic lactam, such as N-vinyl-2-pyrrolidone (“NVP”), or derivatives thereof. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art.

Silicone hydrogels generally have water content greater than about 5 weight percent and more commonly between about 10 to about 80 weight percent. Such materials are usually prepared by polymerizing a mixture containing at least one siloxane-containing monomer and at least one hydrophilic monomer. Typically, either the siloxane-containing monomer or the hydrophilic monomer functions as a crosslinking agent (a crosslinking agent or crosslinker being defined as a monomer having multiple polymerizable functionalities) or a separate crosslinker may be employed. Applicable siloxane-containing monomeric units for use in the formation of silicone hydrogels are known in the art and numerous examples are provided, for example, in U.S. Pat. Nos. 4,136,250; 4,153,641; 4,740,533; 5,034,461; 5,070,215; 5,260,000; 5,310,779; and 5,358,995.

Examples of applicable siloxane-containing monomeric units include bulky polysiloxanylalkyl (meth)acrylic monomers. The term “(meth)acrylic” means methacrylic or acrylic, depending on whether the term “meth” is present or absent. An example of bulky polysiloxanylalkyl (meth)acrylic monomers are represented by the following Formula I:

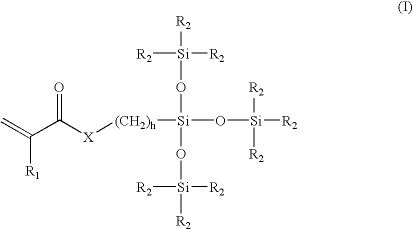

wherein X denotes —O— or —NR—; each R₁ independently denotes hydrogen or methyl; each R₂ independently denotes a lower alkyl radical, phenyl radical or a group represented by

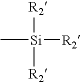

wherein each R′₂ independently denotes a lower alkyl, fluoroalkyl, or phenyl radical; and h is 1 to 10. The term “lower alkyl” means an alkyl radical having 1, 2, 3, 4, 5, 6, 7, 8, 9, or 10 carbon atoms, such as methyl, ethyl, propyl, butyl, isobutyl, pentyl, isopentyl, or hexyl radical.

A suitable bulky monomer is methacryloxypropyltris(trimethyl-siloxy)silane or tris(trimethylsiloxy)silylpropyl methacrylate (“TRIS”).

Another class of representative silicon-containing monomers includes silicone-containing vinyl carbonate or vinyl carbamate monomers such as: 1,3-bis{4-vinyloxycarbonyloxy)but-1-yl}tetramethyld isiloxane; 3-(trimethylsilyl)propyl vinyl carbonate; 3-(vinyloxycarbonylthio)propyl-{tris(trimethylsiloxy)silane}; 3-{tris(trimethylsiloxy)silyl}propyl vinyl carbamate; 3-{tris(trimethylsiloxy)silyl} propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

Another class of representative silicon-containing monomers includes silicone-containing vinyl carbonate or vinyl carbamate monomers such as: 1,3-bis{4-vinyloxycarbonyloxy)but-1-yl}tetramethyl-disiloxane; 3-(trimethylsilyl)propyl vinyl carbonate; 3-(vinyloxycarbonylthio)propyl-{tris(trimethylsiloxy)silane}; 3-{tris(tri-methylsiloxy)silyl} propyl vinyl carbamate; 3-{tris(trimethylsiloxy)silyl} propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula II:

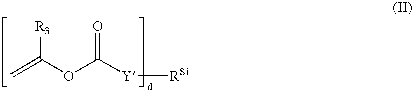

wherein:

Y′ denotes —O—, —S— or —NH—;

R^{Si} denotes a silicon-containing organic radical;

R₃ denotes hydrogen or methyl; and

d is 1, 2, 3 or 4.

Suitable silicon-containing organic radicals R^{Si} include the following:

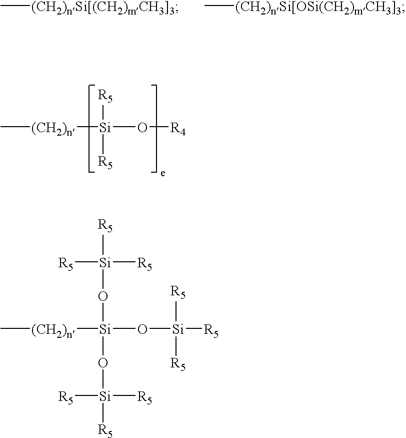

and

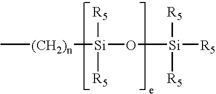

wherein

R₄ denotes

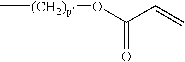

wherein p′ is from 1 to and including 6;

R₅ denotes an alkyl radical or a fluoroalkyl radical having from 1 to and including 6 carbon atoms;

e is 1 to 200; n′ is 1, 2, 3 or 4; and m′ is 0, 1, 2, 3, 4 or 5.

An example of a particular species within Formula II is represented by Formula III.

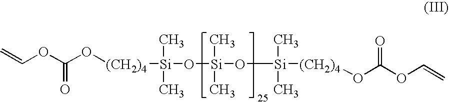

Another class of silicon-containing monomer includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae IV and V:

E(*D*A*D*G)ₐ*D*A*D*E′ (IV)

or

E(*D*G*D*A)ₐ*D*G*D*E′ (V),

wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

denotes a urethane or ureylene linkage;

a is at least 1;

A denotes a divalent polymeric radical of Formula VI:

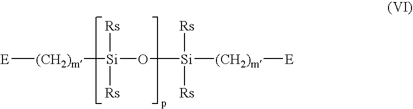

wherein:

each R_{S} independently denotes an alkyl or fluoro-substituted alkyl group having 1 to 10 carbon atoms which may contain ether linkages between carbon atoms;

m′ is at least 1; and

p is a number which provides a moiety weight of 400 to 10,000;

each of E and E′ independently denotes a polymerizable unsaturated organic radical represented by Formula VII:

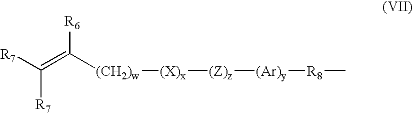

wherein:

R₆ is hydrogen or methyl;

R₇ is hydrogen, an alkyl radical having from 1 to and including 6 carbon atoms, or a —CO—Y—R₉ radical wherein Y is —O—, —S— or —NH—;

R₈ is a divalent alkylene radical having from 1 to and including 10 carbon atoms;

R_{g} is a alkyl radical having from 1 to and including 12 carbon atoms;

X denotes —CO— or —OCO—;

Z denotes —O— or —NH—;

Ar denotes a substituted or unsubstituted aromatic radical having from 6 to and including 30 carbon atoms;

w is from 0 to and including 6; x is 0 or 1; y is 0 or 1; and z is 0 or 1.

A more specific example of a silicone-containing urethane monomer is represented by Formula VIII:

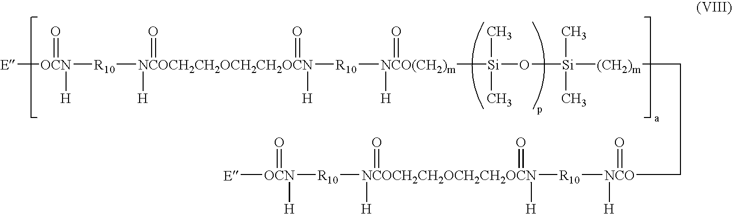

wherein m is at least 1 and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

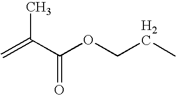

A preferred silicone hydrogel material comprises (in the bulk monomer mixture that is copolymerized) 5 to 50 percent, preferably 10 to 25, by weight of one or more silicone macromonomers, 5 to 75 percent, preferably 30 to 60 percent, by weight of one or more poly(siloxanylalkyl (meth)acrylic) monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

In particular regard to contact lenses, the fluorination of certain monomers used in the formation of silicone hydrogels has been indicated to reduce the accumulation of deposits on contact lenses made therefrom, as described in U.S. Pat. Nos. 4,954,587, 5,079,319 and 5,010,141. Moreover, the use of silicone-containing monomers having certain fluorinated side groups (e.g., —(CF₂)—H) have been found to improve compatibility between the hydrophilic and silicone-containing monomeric units, as described in U.S. Pat. Nos. 5,387,662 and 5,321,108.

In another aspect, a polymeric material of the present invention comprises an additional monomer selected from the group consisting of hydrophilic monomers and hydrophobic monomers.

Hydrophilic monomers can be nonionic monomers, such as 2-hydroxyethyl methacrylate (“HEMA”), 2-hydroxyethyl acrylate (“HEA”), 2-(2-ethoxyethoxy)ethyl (meth)acrylate, glyceryl (meth)acrylate, poly(ethylene glycol (meth)acrylate), tetrahydrofurfuryl (meth)acrylate, (meth)acrylamide, N,N′-dimethylmethacrylamide, N,N′-dimethylacrylamide(“DMA”), N-vinyl-2-pyrrolidone (or other N-vinyl lactams), N-vinyl acetamide, and combinations thereof. Other hydrophilic monomers can have more than one polymerizable group, such as tetraethylene glycol (meth)acrylate, triethylene glycol (meth)acrylate, tripropylene glycol (meth)acrylate, ethoxylated bisphenol-A (meth)acrylate, pentaerythritol (meth)acrylate, pentaerythritol (meth)acrylate, ditrimethylolpropane (meth)acrylate, ethoxylated trimethylolpropane (meth)acrylate, dipentaerythritol (meth)acrylate, alkoxylated glyceryl (meth)acrylate. Still further examples of hydrophilic monomers are the vinyl carbonate and vinyl carbamate monomers disclosed in U.S. Pat. No. 5,070,215, and the hydrophilic oxazolone monomers disclosed in U.S. Pat. No. 4,910,277. The contents of these patents are incorporated herein by reference. The hydrophilic monomer also can be an anionic monomer, such as 2-methacryloyloxyethylsulfonate salts. Substituted anionic hydrophilic monomers, such as from acrylic and methacrylic acid, can also be utilized wherein the substituted group can be removed by a facile chemical process. Non-limiting examples of such substituted anionic hydrophilic monomers include trimethylsilyl esters of (meth)acrylic acid, which are hydrolyzed to regenerate an anionic carboxyl group. The hydrophilic monomer also can be a cationic monomer selected from the group consisting of 3-methacrylamidopropyl-N,N,N-trimethyammonium salts, 2-methacryloyloxyethyl-N,N,N-trimethylammonium salts, and amine-containing monomers, such as 3-methacrylamidopropyl-N,N-dimethyl amine. Other suitable hydrophilic monomers will be apparent to one skilled in the art.

Non-limiting examples of hydrophobic monomers are C₁-C₂₀ alkyl and C₃-C₂₀ cycloalkyl (meth)acrylates, substituted and unsubstituted aryl (meth)acrylates (wherein the aryl group comprises 6 to 36 carbon atoms), (meth)acrylonitrile, styrene, lower alkyl styrene, lower alkyl vinyl ethers, and C₂-C₁₀ perfluoroalkyl (meth)acrylates and correspondingly partially fluorinated (meth)acrylates.

Solvents useful in the surface treatment of the medical device, such as a contact lens, include solvents that readily solubilize the polymers such as water, alcohols, lactams, amides, cyclic ethers, linear ethers, carboxylic acids, and combinations thereof. Preferred solvents include tetrahydrofuran (“THF”), acetonitrile, N,N-dimethyl formamide (“DMF”), and water. The most preferred solvent is water.

### EXAMPLE 1

### Synthesis of Hydroxyl-Containing Poly(Vinylpyrrolidone)

To a 1000-ml three-neck flask equipped with a reflux condenser and nitrogen purge inlet tube were added 900 ml of 2-isopropoxyethanol (about 813.6 g, 7.812 mol), 30 ml of distilled NVP (about 31.3 g, 0.282 mol), 0.32 g (1.93 mmol) of AIBN. The contents were bubbled vigorously with nitrogen for 1 hour. While under moderate nitrogen bubbling and stirring, the contents were heated up to 80° C. for two days. The contents were then heated under vacuum to remove the 2-isopropoxyethanol solvent. Hydroxyl-functionalized poly(vinylpyrrolidone) (“PVP”) was obtained, having a number-average molecular weight of greater than about 1300, as determined by titration.

### EXAMPLE 2

### Preparation of Acrylate-Terminated PVP Polymer

To a thoroughly dried 500-ml round-bottom flask equipped with nitrogen purge inlet tube were added, under a flow of dry nitrogen, 16.95 g (12.49 mmol) of hydroxyl-functionalized PVP produced in Example 2 and 200 ml of anhydrous THF in succession. The flask was cooled with an ice bath. Under stirring, 2.72 g (26.88 mmol) of triethylamine was added to the mixture. Acryloyl chloride (2.333 g, 25.78 mmol) was added dropwise into the mixture. The reaction mixture was warmed up to room temperature and stirred under dry nitrogen for one day. Deionized water (25 ml) was added to the reaction mixture to give a clear solution. Acrylate-terminated PVP as 16.8% (by weight) solution in methanol was recovered using ultrafiltration to remove low molecular weight species. NMR analysis showed broad acrylate function presence in the polymer.

¹H-NMR: 1H: 6.39 ppm, broad doublet; 6.18 ppm, broad multiple; 5.90 ppm, broad doublet; 4.83 single; 4.20 ppm, broad multiple; 3.88 ppm, broad single; 3.740 ppm, broad single; 3.34 ppm, single; 3.30 ppm, multiple; from 2.41 ppm˜1.14 ppm, broad multiple; 0.93 ppm, multiple.

¹³C-NMR: 175.30 ppm, 50.28 ppm, 44.71 ppm, 43.43 ppm, 42.00 ppm, 31.35 ppm, 18.24 ppm.

### EXAMPLE 3

### Surface Treatment of Lenses via the Michael Addition Reaction

PureVision® contact lenses (comprising silicone hydrogel, Bausch & Lomb Incorporated, Rochester, N.Y.) were dried and then plasma treated sequentially with ammonia, butadiene, and ammonia to generate amine-containing groups on the surfaces of the lenses. The lenses were placed in glass vials. Freshly prepared methanol solution containing 16.8% (by weight) of acrylate PVP of Example 2 was then added to the glass vials, which were then set on a rotary machine for three days at room temperature. The treated lenses were rinsed with DI water and stored in borate buffer saline (“BBS”). Control lenses (only plasma treated) were extracted with isopropanol, rinsed with DI water, and placed in BBS. After being desalinated, both control lenses and coated lenses were subjected to standard surface analysis by XPS, and water contact angles were measured on the lenses. The results are shown in Table 1.

TABLE 1
Comparison Between Treated and Control Lenses

| | Contact | | | | |
|---|---|---|---|---|---|
| | Angle | XPS Analysis (atomic percent) | | | |
| Lenses | (degrees) | C₁ₛ | N₁ₛ | O₁ₛ | Si₂ₚ |
| Control | 79.5 (6.3) | 67.7 (2.3) | 5.8 (0.8) | 18.2 (1.2) | 8.4 (1.4) |
| Coated | 49.1 (4.3) | 76.8 (2.2) | 4.5 (0.4) | 16.1 (0.9) | 2.1 (1.3) |

Note:
The numbers in parentheses are standard deviations of three lenses.

The results show an increase in surface carbon and large decreases in surface nitrogen, oxygen, and silicon, indicating that the lens surfaces are covered with the coating polymer. The water contact angle of coated lenses is smaller than that of control lenses, indicating that the coated lenses are more wettable and, thus, should be more lubricious.

In one embodiment, the coated medical device has a water contact angle of less than about 50 degrees. Alternatively, the water contact angle can be less than about 40 degrees, or less than about 30 degrees, or less than about 20 degrees. Low contact angles can be obtained with hydrophilic coating polymers having an abundance of hydrophilic moieties.

### EXAMPLE 4

### Another Surface Treatment of Lenses via Michael Addition Reaction

PureVision® contact lenses (comprising silicone hydrogel, Bausch & Lomb Incorporated, Rochester, N.Y.) were plasma treated in succession with ammonia, butadiene, and ammonia. The plasma-treated lenses were placed in a 5% (by weight) solution of acryloyl chloride in tetrahydrofuran overnight, followed by hydration. The acryloyl chloride-treated lenses were placed in a 2% (by weight) solution of amino-terminated polyamidoamine (“PAMAM”) dendrimer (generation 4) in methanol/water (5/1 v/v) for 72 hours, and then were rinsed with distilled water and stored in borate buffered saline. Another batch of lenses serving as control lenses was not treated with the PAMAM dendrimer, were extracted with isopropanol, then rinses in deionized water and then stored in borate buffered saline.

Both control lenses and PAMAM-treated lenses were desalinated and analyzed by XPS using the standard procedure. Contact angles were also measured on dry lenses following the standard procedure. The results are shown in Table 2.

TABLE 2
Comparison Between PAMAM-Treated and Control Lenses

| | Contact | | | | |
|---|---|---|---|---|---|
| | Angle | XPS Analysis (atomic percent) | | | |
| Lenses | (degrees) | C₁ₛ | N₁ₛ | O₁ₛ | Si₂ₚ |
| Control | 79.5(6.3) | 67.7(2.3) | 5.8(0.8) | 18.2(1.2) | 8.4(1.4) |
| Placed in 5% | 80.9(5.5) | 67.7(2.5) | 3.4(0.7) | 19.3(1.0) | 8.9(1.2) |
| acryloyl chloride/ | | | | | |
| THF overnight | | | | | |
| Placed in 2% | 35.7(3.8) | 71.1(1.0) | 7.2(0.6) | 17.0(0.5) | 4.0(0.4) |
| PAMAM/ | | | | | |
| methanol/ | | | | | |
| Water, 3 days | | | | | |

Note:
The numbers in parentheses are standard deviation of three lenses.

The results show that treatment of PureVision® lenses with hydrophilic PAMAM dendrimer via Michael addition reaction produced a lower contact angle as compared to the control lenses or lenses that were not exposed to PAMAM. The PAMAM-treated lenses also had a significantly lower surface silicon content and a significantly higher surface nitrogen content as compared to control lenses and lenses that were not exposed to PAMAM. The lower contact angle and lower surface silicon content indicate that the PAMAM-treated lenses are much more wettable than control lenses or lenses that were not treated with PAMAM.

The present invention also provides a method for producing a medical device having improved hydrophilic surfaces. In one aspect, the method comprises: (a) providing the medical device having at least a medical-device surface functional group; (b) providing a polymer having at least a hydrophilic moiety and at least a polymer functional group capable of interacting with said at least a medical-device surface functional group through the Michael addition reaction; and (c) contacting the medical device with the polymer at a condition sufficient to produce the medical device having an increased surface hydrophilicity.

In one aspect, the interaction of the polymer functional group and the medical-device surface functional group involves a Michael addition reaction between said groups. In another aspect, such an interaction involves the Michael addition reaction between the polymer functional group and a functional group of a linking compound (or linking polymer), and a second reaction between a second functional group of the linking compound (or linking polymer) and the medical-device surface functional group. In still another aspect, such an interaction involves the Michael addition reaction between the medical-device surface functional group and a functional group of a linking compound (or linking polymer), and a second reaction between a second functional group of the linking compound (or linking polymer) and the polymer functional group.

In one embodiment, the medical device is contacted with the linking compound or polymer and the coating polymer substantially simultaneously. In another embodiment, the medical device may be contacted with the linking compound or polymer in a medium. The coating polymer is subsequently added into the medium after an elapsed time to produce the finally treated medical device.

The step of contacting can be effected at ambient condition or under autoclave condition at about 120° C. The temperature for treatment can range from ambient to about 120° C., or from slightly above ambient temperature to about 80° C. The treatment time can range from about 10 seconds to about 5 days, or from about 1 minute to about 3 days, or from about 10 minutes to about 24 hours, or from about 10 minutes to about 4 hours, or from about 10 minutes to about 2 hours.

In another aspect, the method further comprises the step of treating the surface of the medical device to increase a population of the medical-device surface functional groups before the step of contacting the medical device with the coating polymer or with the coating polymer and the linking polymer. In still another aspect, the step of treating the surface of the medical device is carried out in a plasma discharge or corona discharge environment. In yet another aspect, a gas is supplied to the discharge environment to provide the desired surface functional groups.

Medical devices having a hydrophilic coating of the present invention can be used advantageously in many medical procedures. For example, contact lenses having a hydrophilic coating of the present invention and/or produced by a method of the present invention can be advantageously used to correct the vision of the natural eye.

Medical articles that are in contact with body fluid, such as a wound dressing, catheters, implants (e.g., artificial hearts or other artificial organs), can be provided with a hydrophilic coating of the present invention to inhibit bacterial attachment and growth or to reduce a deposit of lipids or proteins thereon.

In another aspect, the coating polymer of any one of the methods disclosed herein comprises units selected from the group consisting of polymerizable poly(N-vinylpyrrolidone), polyhydric alcohols, polymerizable carboxylic acids, copolymers thereof, combinations thereof, and mixtures thereof.

In a further aspect, the present invention provides a method of making a medical device that has reduced affinity for bacterial attachment. The method comprises: (a) forming the medical device comprising a polymeric material; (b) treating the medical device such that a surface thereof becomes more hydrophilic.

In one embodiment, the method comprises: (a) forming the medical device comprising a polymeric material having at least a medical-device surface functional group; (b) contacting the medical device with a coating polymer having at least a hydrophilic moiety and at least a coating-polymer functional group that is capable of interacting with said at least a medical-device surface functional group via the Michael addition reaction.

In another embodiment, the interaction between the coating polymer and the surface of the medical device is direct. The coating polymer also may interact indirectly with the surface of the medical device through another compound, such as a linking compound or polymer that comprises a first functional group capable of interacting with the medical-device surface functional group and a second functional group capable of interacting with the coating-polymer functional group. One or both of the interactions are effected by the Michael addition reaction.

Non-limiting examples of materials for the medical device, the linking compound or polymer, and the coating polymer are disclosed above.

In one embodiment, the medical device is formed by disposing precursors for the medical device material in a cavity of a mold, which cavity has the shape of the medical device, and polymerizing the precursors.

In another embodiment, a solid block of a polymeric material is first produced, then the medical device is formed from such a solid block; e.g., by shaping, cutting, lathing, machining, or a combination thereof.

In some embodiments, the medical devices produced in a method of the present invention can be contact lenses, intraocular lenses, corneal inlays, corneal rings, or keratoprotheses.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A medical device comprising a surface coating that comprises a coating material that is attached to a surface of the medical device through the Michael addition reaction.

2. The medical device of claim 1, wherein a precursor of the coating material comprises hydrophilic moieties and at least a coating functional group that is capable of interacting with surface functional groups of the medical device via the Michael addition reaction.

3. The medical device of claim 2, wherein when said at least a coating functional group comprises an α-β double bond conjugate to an electron-withdrawing group, said surface functional groups comprise a nucleophilic group; or when said at least a coating functional group comprises a nucleophilic group, said surface functional groups comprise an α-β double bond conjugate to an electron-withdrawing group.

4. The medical device of claim 3, wherein said nucleophilic group is selected from the group consisting of amines, thiols, hydroxyl, hydroxylamines, hydrazines, guanadines, imines, phosphines, carbanions, and combinations thereof.

5. The medical device of claim 3, wherein said nucleophilic group comprises amine functional groups.

6. The medical device of claim 3, wherein said electron-withdrawing group comprises a carbonyl group or a sulfonyl group.

7. The medical device of claim 2, wherein the coating material comprises units derived from a polymerization involving monomers selected from the group consisting of N-vinylpyrrolidone, N,N-dimethylacrylamide, polymerizable alkylene oxides, polymerizable polyhydric alcohols, polymerizable carboxylic acids, saccharides, combinations thereof, and mixtures thereof.

8. The medical device of claim 3, wherein the surface functional groups are created or increased by a method selected from the group consisting of surface implantation of moieties that comprise said functional groups, surface reaction, and combinations thereof.

9. The medical device of claim 3, comprising polysiloxanes.

10. The medical device of claim 9, further comprising units of at least a hydrophilic monomer.

11. The medical device of claim 10, wherein the medical device is a contact lens, intraocular lens, corneal inlay, corneal ring, or keratoprothesis.

12. The medical device of claim 11, wherein the medical device comprises a hydrogel material.

13. The medical device of claim 3, having a water contact angle of less than about 50 degrees.

14. A method for providing increased comfort to a user of a medical device, the method comprising contacting the medical device with a coating polymer comprising units of a polymerizable hydrophilic monomer before using the medical device, wherein the medical device comprises at least a surface functional group, the coating polymer comprises a coating polymer functional group, and said at least a surface functional group and the coating polymer functional group are capable of interacting via the Michael addition reaction.

15. The method of claim 14, wherein said interacting is direct between said surface functional group and said coating polymer functional group.

16. The method of claim 14, wherein said interacting is through a linking compound and said Michael addition reaction is effected between said surface functional group and said linking compound or between said linking compound and said coating polymer functional group.

17. The method of claim 14, further comprising increasing a population of the surface functional group before the step of contacting.

18. The method of claim 17, wherein the step of increasing the population of the surface functional group is effected by a method selected from the group consisting of surface implantation of moieties that comprise said functional groups, surface reaction, and combinations thereof.

19. The method of claim 17, wherein the step of increasing the population of the surface functional groups is carried out in a plasma discharge or a corona discharge environment.

20. The method of claim 14, wherein when said coating polymer functional group comprises an α-β double bond conjugate to an electron-withdrawing group, said at least a surface functional group comprises a nucleophilic group; or when said coating polymer functional group comprises a nucleophilic group, said at least a surface functional group comprises an α-β double bond conjugate to an electron-withdrawing group.

21. A method for making a medical device having an increased surface hydrophilicity, the method comprising:
(a) providing the medical device comprising a polymeric material having at least a surface functional group;
(b) providing a coating polymer having at least a coating polymer functional group capable of interacting with said at least a surface functional group via the Michael addition reaction; and
(c) contacting the medical device with the coating polymer at a condition sufficient to carry out the Michael addition reaction to produce the medical device having an increased surface hydrophilicity.

22. The method of claim 21, further comprising the step of increasing a population of the surface functional group before the step of contacting.

23. The method of claim 22, wherein the step of increasing the population of the surface functional group is carried out in a plasma discharge or a corona discharge environment.

24. The method of claim 21, wherein the step of providing comprises forming the medical device from the polymeric material.

25. The method of claim 21, wherein the medical device is selected from the group consisting of contact lenses, intraocular lenses, corneal inlays, corneal rings, and keratoprotheses.

26. The method of claim 21, wherein said at least a surface functional group is selected from the group consisting of amines, thiols, hydroxyl, hydroxylamines, hydrazines, guanadines, imines, phosphines, carbanions, and combinations thereof.

27. The method of claim 21, further comprising contacting the medical device with a linking compound before or substantially simultaneously with contacting with the coating polymer.

28. The method of claim 27, wherein the Michael addition reaction is effected between the surface functional group and the linking compound.

29. The method of claim 27, wherein the Michael addition reaction is effected between the coating polymer functional group and the linking compound.

30. The method of claim 20, wherein when said at least a coating polymer functional group comprises an α-β double bond conjugate to an electron-withdrawing group, said at least a surface functional group comprises a nucleophilic group; or when said at least a coating polymer functional group comprises a nucleophilic group, said at least a surface functional group comprises an α-β double bond conjugate to an electron-withdrawing group.
